(19) United States
(12) Patent Application Publication
(10) Pub. No.: US 2010/0129932 A1
(43) Pub. Date: May 27, 2010

(54) Determination of Isoerythropoietins

(76) Inventors: Maria Lönnberg, Knivsta (SE); Jan Carlsson, Uppsala (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/593,790

(22) PCT Filed: Apr. 21, 2008
(86) PCT No.: PCT/SE08/00276

§ 371 Date: Sep. 29, 2009

Related U.S. Application Data
(60) Provisional application No. 60/943,857, filed on Jun. 14, 2007.

(30) Foreign Application Priority Data
Jun. 5, 2007 (SE) 0701457-4

(51) Int. Cl. G01N 33/53

(52) U.S. Cl. 436/501

(57) ABSTRACT

A method for the determination of the occurrence of an analyte subpopulation of isoEPOs in a biological fluid comprising the steps of: i) providing a liquid sample deriving from the biological fluid and containing isoEPOs that are characteristic (=analyte isoEPOs) for said analyte subpopulation together with isoEPOs of other subpopulations, ii) separating the isoEPOs of said liquid sample into two or more fractions so that at least one of these fractions contains analyte isoEPOs which are enriched relative to other isoEPOs, by A) contacting the liquid sample with an isoEPO reactant under conditions allowing affinity complex formation between isoEPOs of the liquid sample and the affinity reactant, and B) fractionating and collecting the isoEPOs of the liquid sample into said two or more fractions, relating the occurrence of analyte isoEPOs in said at least one fraction to the occurrence of said analyte subpopulation in the biological fluid. A flow matrix containing an application zone and downstream thereof a separation zone in which there is an immobilized isoEPO reactant.

### TECHNICAL FIELD

The invention relates to a method for the determination of the occurrence of a desired subpopulation of isoerythropoietins (isoEPOs) in a biological fluid, primarily of a mammal. In another aspect the invention is a flow matrix that can be used in the method. The mammal is typically a human or a domestic mammal.

### DEFINITIONS

Isoerythropoietins (isoEPOs) are proteins that are structurally related to erythropoietin (EPO) which might be capable of affecting erythropoiesis, e.g. by stimulating the production of erythrocytes, affecting angiogenesis etc. Their polypeptide backbones are in essence the same, thus including variants that are produced by a) posttranslational modification, such as deamidation, addition of carbohydrate structures, phosphorylation, sulphonation etc, b) recombinant techniques for replacement, deletion and/or addition of one, two, three, four, five or more amino acid residues, c) chemical modification, e.g. fragmentation, addition of various groups to the polypeptide backbone or to a carbohydrate moiety. EPOs according to b) and c) will be called EPO-analogues.

A “subpopulation of isoEPOs” is a selected group of isoEPOs and may comprise a single isoEPO or a combination of two, three or more different isoEPOs. Primarily “subpopulation” refers to isoEPOs having a common origin, for instance produced in the kidney, liver, brain etc, or recombinantly. Various kinds of recombinantly produced EPOs are considered as separate subpopulations, e.g. differently mutated variants, variants having the same polypeptide backbone but produced in different kinds of cells. IsoEPOs that are characteristic for a certain disease are also considered as a separate subpopulation.

The subpopulation to be determined will be called “analyte subpopulation” or “desired subpopulation”. IsoEPOs which define at least a part of the characteristic isoform pattern of an analyte subpopulation and which are to be used for determining if the analyte subpopulation is present in the biological fluid of interest will be called “analyte isoEPOs” or “desired isoEPOs”.

If not otherwise indicated the term EPO will be used generically.

All US patents and patent applications cited in this specification are hereby incorporated by reference in their entirety.

### TECHNICAL BACKGROUND

Erythropoietin (EPO) is a 30 kD sialoglycoprotein hormone that stimulates production of new red blood cells (erythropoiesis) in mammals. In adults, EPO is mainly produced by certain renal cells and in less amounts also in the liver (<10%) and in the brain. The stimulus for initiating or enhancing EPO production comprises tissue hypoxia that in most cases is related to the number of circulating erythrocytes and sensed by tissue in which EPO-producing cells are present. Soon after recombinant human EPO became available in the mid-eighties the protein was used as a replacement agent in patients with impaired production of EPO and for improving performance in endurance sports. In spite of the lack of analytical methods for discriminating endogenous from exogenous EPO in samples from sportsmen, the substance was quickly classified as a doping agent and banned by international sport federations.

The glycan chains of EPO constitute about 40% of its molecular weight and are attached to Asn24, 38 and 83 and to Ser126. As many other circulating glycoproteins EPO is extremely heterogeneous with respect to carbohydrate content. Based on techniques revealing differences in charges the number of isoforms has been estimated to be about 30-50 and is strongly dependent on the enzymatic system provided for glycosylation by the cells that produce EPO. Thus the isoform pattern for renal, liver and various recombinant EPOs differ significantly from each other. The differences in isoform pattern of EPO between different samples have been the basis for most attempts to determine the origins of the EPO isoforms in individual samples. Typically the methods have utilized differences in sialyl group content and comprised a first step in which EPO of a sample, e.g. a urine or a serum sample, is concentrated followed by electrophoresis, in particular isoelectric focusing (IEF), of the concentrated EPO in order to separate the various isoforms from each other and to determine abnormal deviations in the isoform pattern found. Presteps for concentrating EPO have mainly utilized ultrafiltration and various affinity adsorbents such as adsorbents carrying a lectin or an anti-EPO antibody as the capturing ligand. Labelled lectins have been suggested for probing isoEPOs after IEF. Deviations searched for have been the occurrence of isoforms that a) derive from exogeneous EPO that has been administered to the individual, e.g. various recombinant forms, or b) are disease-related. See further:
Amadeo G I et al., Braz. J. Chem. Eng. 20:1 (March 2003) 21-26;
Fraguas L F et al., Interlec21 (International Lectin meeting) 23-28 May 2004, Kanagawa, Japan (Abstract published in Trends in Glycoscience and Glycotechnology (TIGG) 16 (2004) 563);
Robinson et al., Br. J. Sports Med. 40 (July 2006) i30-i34;
Lasne et al., Nature Vol 405 (8 Jun. 2000) 635;
Lasne et al., Anal. Biochem. 311 (2002) 119-126;
Nagano M et al., Electrophoresis 26 (2005) 1633-1645;
Spivak et al., Blood 52:6 (1978) 1178-1188;
Wide L et al., Brit. J. Haematol. 76 (1990) 121-127;
Wide et al., Med. Sci. Sports Exerc. 27:11 (1995) 1569-1576;

The assays so far approved are time-consuming, expensive and require highly trained and specialized laboratories. There is a need for simplified assays. Complications have been the complex isoform patterns that are obtained for endogeneous as well as for recombinantly produced EPOs. Due to overlapping, the complexity is enhanced if recombinantly produced variants of EPO are present together with endogenous variants, such as in samples deriving from biological fluids of individuals suffering from a disease affecting the isoform pattern and/or to which recombinant variants have been administered therapeutically or as a doping agent. It has therefore been considered more or less impossible to base reliable clinical assays on chromatographic techniques separating EPO variants from each other for a reliable qualitative and quantitative determination of a clinically relevant subpopulation in a parent sample containing also other subpopulations, e.g. disease-related or non-endogenous. Immunologically the variants are too similar to easily allow for simple immunoassays of a certain variant in the presence of other variants.

The problems encountered for discriminating between subpopulations of EPO has been far more complex than the problems associated with discriminating between clinically relevant isoforms of other sialoglycoproteins for which useful methods many times have been around for 10-20 years. For transferrin see Cervén E et al., WO 1982000204; Joustra et al., WO 1985003758.

Chromatographic techniques for isolation of EPO from non-clinical samples of either native or recombinant origin, and fractionation of the EPO preparation so obtained into subpopulations containing various combinations of isoEPOs have been around for quite a long time. See e.g. Gokana et al. (J. Chromatog. A 791 (1997) 109-118) and Spivak et al. (Proc. Natl. Acad. Sci. USA 74 (1977) 4633-4635).

### OBJECTIVES

The primary objective is to provide improved methods within the field defined in the introductory part.

### INVENTION

The inventors have realized that the problems discussed above can be at least partially circumvented if isoEPOs that are characteristic of the subpopulation of interest are enriched relative to other isoEPOs by the use of a reactant that is an affinity counterpart to EPO and has an affinity for EPO that varies with different isoEPOs. An important contribution to the invention is the inventors' unexpected discovery that even if a carbohydrate specific affinity reactant quantitatively binds all isoforms of EPO of a biological fluid, the reactant can be used to discriminate between subpopulations by selecting the appropriate reaction conditions (e.g. desorbing conditions). By applying this principle to selectively dissociate isoEPOs, which are characteristic for a certain subpopulation from the affinity complex formed, it has been possible to reliable characterize the presence of this subpopulation in a biological fluid that also contains other subpopulations of EPO.

A first main aspect of the invention is a method for the determination of the occurrence of a desired subpopulation (analyte subpopulation) of EPO in a biological fluid, typically of a mammal. In its broadest sense the method comprises at least three steps:
i) Providing a liquid sample that derives from the biological fluid and contains isoEPOs that are characteristic (=analyte isoEPOs) for the analyte subpopulation together with other isoEPOs, e.g. of other subpopulations.
ii) Separating the isoEPOs in the liquid sample into two or more fractions so that at least one of these fractions contains analyte isoEPOs which are enriched relative to other isoEPOs, e.g of other subpopulations, by
   A) contacting the liquid sample with an isoEPO specific affinity reactant (=isoEPO reactant) under conditions allowing affinity complex formation to different degree for the different complexes formed between the various isoEPOs of the liquid sample and the affinity reactant,
   B) fractionating and/or capturing (=saving, collecting) the isoEPOs of the liquid sample into said two or more fractions as a function of said variation in affinity,
iii) relating the occurrence of the analyte isoEPOs in said at least one fraction to the occurrence of the analyte subpopulation in the biological fluid.

Substep (ii.A) includes allowing complex formation to take place.

Substeps (ii.A) and (ii.B) may coincide or may be distinct steps, preferably with substep (ii.A) preceding substep (ii.B). “Different degree” above refer to the differences in affinity constant and/or differences in rates of formation/association and/or dissociation (=tendencies in formation and dissociation, respectively), i.e. involving differences in behaviour between analyte isoEPOs and other isoEPOs (see below).

The method includes that steps (ii) to (iii) is performed for the determination of one, two or more different analyte subpopulations by relating the occurrence of different sets of analyte isoEPOs to different sets of fractions (i.e. the at least one fraction varies for the different analyte subpopulations) and/or by repeating steps (i)-(iii) with different isoEPO reactants and/or different conditions in step (ii.A).

The analyte subpopulation is primarily one of those mentioned under “Definitions” above.

### The Liquid Sample (Step i)

When transforming a sample of a biological fluid to a liquid sample to be used in step (i) and to the at least one fraction enriched in analyte isoEPOs, it is important to select the conditions so that the amount of the analyte isoEPOs in the liquid sample and in the at least one fraction will be a function of the amount of these isoEPOs in the biological fluid.

Step (i) typically comprises processing a sample of a biological fluid of interest to a sample in which the concentration of EPO is increased, e.g. in absolute terms (amount/volume etc), enriched relative to other proteins, in particular relative to other glycoproteins etc. Fluids of interest contain EPO, such as whole blood, urine, fluids deriving from recombinant production of EPO, etc. Whole blood samples thus typically are first processed to serum or plasma samples, and if required diluted with the appropriate buffer. Step (i) typically also comprises concentrating a primary sample of the biological fluid of interest with respect to EPO, e.g. a plasma or serum or urine sample. Concentrating may be accomplished by ultrafiltration or by the use of various affinity adsorbents exposing ligands with broad specificity for EPO, i.e. with no significant discrimination between various isoEPOs. Useful ligands are anti-EPO antibodies that a specific for epitopes defined by the polypeptide backbone as well as lectins that are capable of affinity binding to carbohydrate structures that are present in most isoforms of EPO. See for instance Amadeo et al cited above. If the sample contains proteolytic activity, e.g. urine samples, it is preferred to add protease inhibitors prior to the processing.

In one variant of step (i) only a certain portion of the isoEPOs of the biological fluid is concentrated. IsoEPOs having isolectric points pIs within a certain interval may thus be selectively concentrated by isoelectric focusing (IEF) or by ion-exchange chromatography. For the latter technique see for instance U.S. Pat. No. 6,902,889, U.S. Pat. No. 6,737,278, U.S. Pat. No. 6,528,322, and US 20040023412 (all Carlsson, J & Lönnberg, M)and WO 1985003578 (Joustra et al). See also Wide at al (1990) cited above.

### Separation Step (Step ii)

This step typically results in fractions of isoEPOs that are physically separated from each other. The isoEPOs of a fraction may be present either complexed or not complexed to the isoEPO reactant. If complexed the complex may be dissociated in a subsequent step to provide a fraction in which the isoEPOs are uncomplexed. The complexed isoEPOs of a fraction may be in dissolved or insolubilized form as discussed below.

The separation conditions are typically selected to enable formation of an affinity complex in aqueous media between the isoEPO reactant and the various isoEPOs. In a first variant the initial conditions including the isoEPO reactant are selected to promote direct selective enrichment of the analyte isoEPOs in the uncomplexed or in the complexed portion of isoEPOs. “Direct” here means in one step. Fractionation is then accomplished by physically separating the portion enriched in analyte isoEPOs from the other portion. In a second variant the corresponding initial conditions are selected to promote that essentially all of the EPOs irrespective of being analyte-isoEPOs or non-analyte isoEPOs are incorporated in the complex. Fractionation is thereafter accomplished by changing the conditions in the medium in contact with the complex to promote partial dissociation of the complex and divide the EPOs into one portion containing complexed isoEPOs (complexed portion) and another portion containing uncomplexed isoEPOs (first uncomplexed portion), and then separating the two portions from each other. A second portion of uncomplexed isoEPOs can be formed and removed as a second uncomplexed fraction etc by extended contact under the same or different conditions after the first uncomplexed portion/fraction has been removed. The fraction enriched in analyte isoEPOs are collected and used in step (iii). The change in conditions typically involves inclusion of an increasing concentration of a dissociating agent to the liquid in contact with the complex. This also includes that the kind of dissociating agent may be changed, for instance from a weaker to a more efficient dissociating agent. Suitable agents are typically low molecular weight soluble compounds that mimic the structure the isoEPO reactant is specific for, e.g. including an inhibitor for complex formation . In the case the isoEPO reactant is immobilized this agent is also called desorbing agent. The molecular weights of suitable dissociating/desorbing agents are typically <5 kD, such as <2 kD. Other separation formats involving formation of affinity complexes can be envisaged, e.g. based on different rates for complex formation (association) for the various isoEPOs leading to the possibility that the physical separation of uncomplexed and complexed portions can take place before the complex reaction has reached equilibrium.

In preferred variants the isoEPO reactant is insolubilized or insolubilizable and placed in contact with a liquid phase that initially contains isoEPOs of the original sample. Insolubilized/insolubilizable typically means immobilized to a solid phase. The advantages with insolubilization are that physical separation of the uncomplexed from the complex-bound portion is facilitated.

Steps (ii.A) and (ii.B) may in variants of the preceding paragraph at least partially coincide.

Steps (ii.A) and (ii.B) then preferably comprise the substeps of:
a) adjusting the conditions in the liquid phase/sample to promote selective complexation of analyte isoEPOs or of non-analyte isoEPOs with the immobilized isoEPO reactant thereby enriching analyte isoEPOs to the solid phase in complexed form or to the liquid phase in uncomplexed form, respectively, and
b) capturing (=saving) the isoEPOs complexed on the solid phase or uncomplexed in the liquid phase as said at least one fraction enriched in analyte isoEPOs.

Partial complexation as discussed for this variant may be accomplished by including an inhibitor of the type discussed elsewhere in this specification in the liquid phase in contact with the solid phase during formation of the complex and/or by performing step (b) before complex formation has reached equilibrium.

Steps (ii.A) and (ii.B) may alternatively be non-coinciding (distinct) in other variants utilizing an insolubilized or insolubilizable isoEPO. Step (ii.B) in these other variants may comprise the substeps of:
a′) adjusting the conditions provided by the liquid phase in contact with the immobilized affinity complex such that analyte isoEPOs are selectively dissociated/released from or selectively retained by the isoEPO reactant on the solid phase thereby enriching analyte isoEPOs to the liquid phase in uncomplexed form or to the solid phase in complexed form, respectively, and
b′) capturing (=saving) the isoEPOs complexed on the solid phase or uncomplexed in the liquid phase as said at least one fraction enriched in analyte isoEPOs.

This variant may comprise further fractionation substeps:
a″) adjusting the conditions provided by the liquid phase in contact with the solid phase to which the affinity complex is immobilized such that isoEPOs of none, one, two, three or more subpopulations other than said analyte subpopulation are dissociated/released from the complex/solid phase, and
b″) capturing (=saving) these other released isoEPOs as fractions of the remaining ones of said two or more fractions.

Substeps (a″) and (b″) may be carried out in a pairwise/stepwise fashion with one or more rounds of adjustment-saving taking place prior to and/or subsequent to substeps (a′) and (b′) with preference for at least prior to when substeps (a′) and (b′) means that analyte isoEPOs in complexed form are retained on the solid phase.

The isoEPOs saved according to steps b), b′) and b″) in the variants of the preceding paragraphs may be subjected to further fractionation to obtain the actual fractions used in step (iii).

Complex formation can take place under static non-flow conditions, i.e. batch procedures, or under flow conditions. Flow conditions in this context means that the isoEPOs are transported in a liquid flow relative to the affinity reactant that typically is at a fixed position, e.g. immobilized to a solid phase. Flow conditions are for instance at hand when chromatographic conditions are applied to step (ii.A) and (ii.B) with the isoEPO reactant immobilized to a porous solid phase through which the isoEPOs are transported by a liquid flow (i.e. the solid phase is a flow matrix).

During step (ii.A) the conditions provided by the liquid phase are typically isocratic with respect to variables that are critical for complex formation. In flow systems that utilize an isoEPO reactant that is immobilized to a flow matrix isocratic conditions typically will promote concentratring of isoEPOs to an upstream portion of a solid phase containing the isoEPO reactant. This will in particular be important for samples of relatively large volumes in which the concentration of isoEPOs are low. This does not exclude the possibility of using non-isocratic conditions during step (ii.A), e.g. for small volume samples in which EPO is present in larger concentrations.

During step (ii.B) the conditions provided by the liquid phase are typically non-isocratic with respect to variables that facilitates dissociation and fractionation of the isoEPOs into a complexed fraction and an uncomplexed fraction. In flow systems that utilize an reactant that is immobilized to a flow matrix, non-isocratic conditions typically will favour a more efficient fractionation process with respect to placing isoEPOs of different subpopulations into different fractions. This does not exclude that isocratic conditions may be used in step (ii.B), such as when this step at least partially coincides with step (ii.A) in flow systems utilizing a flow matrix.

During isocratic conditions variables that are critical for complex formation and complex dissociation are kept constant. During non-isocratic conditions at least one such variable is changed as a gradient. Typical such variables comprise concentration and/type of dissociating agent. See above. The change/gradient may be stepwise, e.g. with two, three or more steps, or continuous.

The isoEPO Reactant

An isoEPO reactant is an affinity reactant that can discriminate between different isoEPOs, i.e. analyte isoEPOs from other isoEPOs. The discrimination may relate to differences in affinity constants, and/or in dissociation rates and/or association rates of an affinity complex that contains both the reactant and an isoEPO.

With the present knowledge, potentially useful affinity reactants are primarily selected amongst lectins that are capable of binding to carbohydrate structures that are present in EPO, preferably N-acetyl glucose amine structural units. As illustrated in the experimental part wheat germ agglutinin (WGA) is at the filing of this specification considered to be the best iso-EPO specific affinity reactant.

The term “lectin” comprises plant proteins that have biospecific affinity for a carbohydrate structure. This includes also native lectins that have been modified, for instance by chemical or recombinant techniques. Entities of non-plant origin that mimics the carbohydrate binding ability of native lectins, for instance anti-carbohydrate specific antibodies and fragments and mimetics thereof are also lectins in the context of the invention.

When screening for affinity reactants that are capable of discriminating between isoEPOs of different subpopulations according to affinity as required by the invention, it is important to combine such a screening with a screening for optimizing other variables applied in steps (ii.A) and (ii.B). Variables of particular interest are a) kind of affinity reactant and its concentration, e.g. on a solid phase, b) kind of desorbing agent and its concentration in a desorbing solution (including suitable gradients), c) flow rates (applicable in particular when the affinity reactant is immobilized to a solid phase, e.g. chromatographic procedures), d) reaction time (e.g. in static system=non-flow conditions), e) insolubilization/immobilization technique etc. Variables that also may be of interest are: pH, solvent composition, ionic strength, etc. This kind of screening follows well-known principles. Similar principles have been utilized by Fraguas L F et al (cited above).

Insolubilization of the isoEPO Reactant and Solid Phases

The isoEPO reactant is preferably provided in insoluble form prior to step (ii.A). Alternatively the isoEPO reactant may be provided in insolubilizable form (soluble/dissolved form) at the start of step (ii.A) which means that the affinity complex is formed in dissolved form and during the step preferably is transformed to an insoluble form before the actual fractionation is taking place. In preferred variants the terms insoluble/insolubilizable/insolubilization refer to immobilized/immobilizable/immobilization of the affinity reactant to a solid phase.

Solid phases are well known in the field and encompass surfaces, such as inner surfaces of inner walls of reaction vessels, particles, for instance in the form of beads, which may be porous or non-porous, porous monolithic plugs, membranes, sheets etc. Particles may be in suspended form or in the form of packed beds/sedimented beds.

The material in the solid phase, e. g. in particles, is typically polymeric, for instance a synthetic polymer or a biopolymer and includes also inorganic polymers such as glasses. Solid phases (e.g. particles packed to a bed) are typically hydrophilic in the sense that they will be saturated by water by the action of capillarity (self-suction) if in contact with an excess of water. The term also indicates that the surfaces of the solid phase material shall expose a plurality of polar functional groups each of which comprises a heteroatom selected amongst oxygen, sulphur, and nitrogen.

Particularly preferred solid phases are those that in addition to being carrier for the reactant and facilitating separation of complexed from uncomplexed isoEPOs also can function as flow matrices. A flow matrix typically is defined as the internal surface of a single flow channel (for instance of capillary dimensions), the internal surface of a porous matrix having a penetrating system of flow channels (porous matrices) etc. A flow matrix may be in the form of a monolith, sheet, column, membrane, separate flow channels, or aggregated systems of flow channels. The flow matrices may also be in the form of particles packed in column cartridges or in cut grooves, compressed fibres etc. See further U.S. Pat. No. 6,902,889, U.S. Pat. No. 6,737,278, U.S. Pat. No. 6,528,322, and US 20040023412 (all Caisson, J & Lönnberg, M).

Another interesting flow matrix to be used in the invention is designed as a laterally extending microstructured surface area in a planar material comprising microprojections extending substantially perpendicular to the surface and at a sufficient short distance from each other to provide capillary transport (self-suction) of an aqueous liquid such as water which is placed in liquid contact with a spot defined in the microstructured surface area. See for instance WO/2007/149043, WO 2007/149042, 2006/137785, WO 2005/118139; 2005089082 (all of Åmic AB).

The techniques for immobilization may be selected amongst those that are known in the field, for instance via covalent bonds, affinity bonds (for instance biospecific affinity bonds), physical adsorption (mainly hydrophobic interaction) etc. Examples of bioaffinity bonds that can be used are bonds between individual members of a bioaffinity pair such as avidin/streptavidin/neutravidin etc and biotin or biotin derivatives, a high affinity antibody and a hapten or a derivative of the hapten, etc. where one member of the pair is linked to the solid phase and the other to the isoEPO reactant. Examples of other affinity bonds are between polar groups or charged groups on the solid phase and polar groups and charged groups on an isoEPO reactant (includes electrostatic bonds), between hydrophobic groups on the solid phase and hydrophobic groups on an isoEPO reactant. If the appropriate immobilizing affinity group is not inherently present on a solid phase or an isoEPO reactant such a group may be introduced by derivatization (chemically, recombinantly etc).

Many times it is advantageous to immobilize the isoEPO reactant to the solid phase via a carrier molecule to which one, two, three or more molecules of the isoEPO reactant are covalently attached (per carrier molecule). The carrier molecule may inherently contain the groups that are necessary for its immobilization to the solid phase or is derivatized to contain such groups. These groups may provide for immobilization via covalent bonds or affinity bonds of the types discussed in the preceding paragraph. In preferred variants the bonds between the isoEPO reactant and the carrier are covalent while affinity bonds are utilized for attaching the carrier to the solid phase. The carrier typically comprises polymer structure and provides multipoint attachment to the solid phase simultaneously with being a carrier for two or more molecules of the isoEPO reactant (per carrier molecule). Suitable carriers shall be inert towards the intended reaction, i.e. the affinity reaction between isoEPOs and the isoEPO reactant, and may comprise polypeptide structure, e.g. be an albumin such as serum albumin, or comprise other kinds of polymer structure, e.g. exhibiting a plurality of hydroxyl and/or amide and/or amine groups and if required derivatized to exhibit affinity groups of the types discussed above.

The isoEPO reactant is in a flow matrix preferably immobilized in a separation zone (SZ) downstream of an application zone (AZ). The separation zone can be used for carrying out step (ii) by transporting the isoEPOs of the liquid sample through the zone by a liquid flow. A detection zone (DZ) may be incorporated in the flow matrix by immobilizing a capturer for the appropriate reactant of the assay used in step (iii) in a zone downstream of the separation zone (SZ). A flow path 1 is defined between the application zone (AZ) and the detection zone (DZ).

A flow matrix may contain additional application zones (AZ₂,AZ₃,AZ₄ etc) and/or detection zones (DZ₂,DZ₃,DZ₄ etc) with additional flow paths 2,3,4 etc where detection zones respective flow paths may partly or fully coincide. These additional application zones may be used for addition of reactants other than EPO, e.g. a detectable reactant for carrying out step (iii) (see below). Three main variants are
A) an application zone AZ₂ that is placed between a sample application zone AZ₁ and a detection zone DZ₁ defining a flow path 2 that from AZ₂ coincides with the downstream part of flow path 1, or
B) an application zone AZ₃ that together with a detection zone DZ₃ defines a flow path 3 that is separate from flow path 1 with a flow direction which is (a) transversal, or (b) opposite to the flow direction of flow path 1 (as in FIG. 3b), e.g. DZ₃ is separate or coincides, respectively, with a detection zone DZ₁, or
C) an application zone AZ₄ that coincides with a detection zone DZ₁ with transport out of the zone of excess of detectable reactant after reaction in the zone.

For alternative (A) it is preferred that the part of the flow matrix/flow path that is upstream of application zone AZ₂ is removable. Alternative (B.a) is in particular useful in the case further fractionation is to take place of an isoEPO fraction that remains in the separation zone after a first fraction has been desorbed along flow path 1 to detection zone DZ₁ (example of variants in which the two flow paths intersect each other at SZ₁). Provided different isoEPO fractions are located at different longitudinal positions in the separation zone it may be beneficial to have several parallel flow paths that are transversal to flow path 1 and passing the separation zone of flow path 1. Each such parallel flow path may contain its own SZ and/or DZ downstream of the separation zone of flow path 1 and an AZ in its upstream part.

In an alternative kind of flow matrices the separation zone and the detection zone may coincide. This kind of flow matrices may in particular be useful for methods of the invention in which analyte isoEPOs after fractionation in step (ii.B) remain on the solid phase, i.e. in the separation zone. Application zones may be placed as described above.

The flow matrix may contain a plurality of completely separate and identical flow paths in order to carry out several different and/or identical runs of the method of the invention in parallel. The kind, amount and distribution of the isoEPO reactant in the separation zone may differ between the flow paths.

The material in the different parts of the flow matrix may differ dependent on function of the part. Thus the material in the reaction zones (i.e. separation zones and/or detection zones) may differ from the material in transport zones TZs. Examples of transport zones are zones between a) an application AZ and a separation zone SZ, b) a separation zone SZ and a detection zone DZ, c) different detection zones, d) different separation zones SZs, d) a detection zone DZ and the outlet end of the flow matrix (“waste”).

These kinds of systems may be in the form of test strips in which the porous matrix is in the form of a porous sheet. The porous sheet is typically placed on a plastic backing that is impermeable for the liquid used to transport reactants.

What has been said above about preferences for immobilization to solid phases of the isoEPO reactant in particular applies to solid phases in the form of a flow matrix.

More details about suitable flow matrixes and arrangements of various zones are given in U.S. Pat. No. 6,902,889, U.S. Pat. No. 6,737,278, U.S. Pat. No. 6,528,322, and US 20040023412 (all Carlsson, J & Lönnberg, M).

Relating occurrences in fractions to the biological fluid (step (iii))

This step comprises the substeps of:
a) measuring the amount of analyte isoEPOs in one or more of the above-mentioned at least one fraction, and
b) relating this amount to the occurrence of analyte subpopulation in the biological fluid of interest.

The measurement in substep (iii.a) above may be direct or indirect. Direct means that the amount of the isoEPOs in the actual fractions is measured. Indirect means that the amount of isoEPOs in fractions other than the at least one fraction enriched in analyte isoEPOs is measured and that the values obtained are used to calculate analyte isoEPOs in the at least one fraction.

The terms “amount” and “level” are used interchangeable.

The amount in substep (iii.a) may be the presence or absence of analyte isoEPOs in the one or more fractions which for substep (iii.b) will mean occurrence and non-occurrence of the analyte subpopulation in the biological fluid. In other variants the actual concentration and/or the amount of analyte isoEPOs, or more preferable relative amounts are measured, i.e. the absolute amount of analyte isoEPOs relative to the amount of an internal standard. Preferred internal standards are:
A) the amount of one or more isoEPOs that are a) present in the liquid sample, b) different from the analyte isoEPOs measured in step (iii.a) and c) characteristic of the analyte subpopulation and/or of one or more other subpopulations that are present in the liquid sample, or
B) total amount of isoEPOs (=EPO) in the liquid sample.

Typical other subpopulations of (A) are the aberrant isoEPOs discussed in the experimental part, disease-related isoEPOs, kidney isoEPOs or liver isoEPOs etc. The measurement of the relative amount of analyte isoEPOs can be combined with the measurement of the relative amount of isoEPOs that are different from the analyte isoEPOs measured in step (iii.a) and characteristic for the analyte subpopulation or for one or more other (e.g. non-analyte) subpopulations.

There are further variables in addition to amounts that reflect differences in affinity between the analyte isoEPO and the mean affinity of the isoEPOs of the liquid sample and therefore can be measured in step (iii.a). If the separation step is carried out in a flow matrix as illustrated in the experimental part and the liquid sample contains elevated relative levels of analyte isoEPOs that adsorb stronger or weaker than other isoEPOs of the liquid sample, then the following variables are of interest:
a) the elution volume and/or concentration of a desorption agent required to desorb a predetermined portion (e.g. 50%) of the total amount of isoEPOs and/or
b) the relative amount desorbed for a fixed elution volume or a fixed concentration of a particular desorption agent.

If the adsorption for analyte isoEPOs is stronger than for other isoEPOs, larger volumes and/or higher concentrations of desorption liquid/agent will be required.

The measurement of isoEPOs used as standard is typically carried out by measuring them in the appropriate fractions optained in step (ii). Total amount of EPO may be measured in the liquid sample without fractionation or as the sum of EPO in all fractions containing isoEPOs.

Some variants of the invention comprises that the amount of isoEPOs in every fraction containing isoEPOs is measured relative to the total amount of EPO and represented as an elution profile which is compared with a mixture simulated to contain the analyte subpopulation together with the normal subpopulation(s) of the biological fluid. By applying pattern recognition on the elution profile the amounts of individual subpopulations, e.g. an analyte subpopulation can be determined.

Fractions containing the isoEPOs to be measured in step (iii.a) may be pooled, e.g. fractions containing analyte isoEPOs or the aberrant isoEPOs discussed in the experimental part, etc.

The measuring step (iii.a) may be preceded by further fractionation of the fractions enriched in analyte isoEPOs into subfractions. In this case the measurement of EPO can be performed in one or more of these subfractions. Further fractionation of a fraction that is in the form of a liquid aliquot in which the isoEPOs are uncomplexed and dissolved may include fractionation with a second isoEPO affinity reactant that is capable of discriminating between isoEPOs of the fraction. In general terms the protocol used may be the same as or similar to the protocol used in step (ii). Completely different fractionation protocols may also be used such as ion exchange chromatography, electrophoresis, in particular isoelectric focusing (IEF). Further fractionation of a fraction in which the isoEPOs are in complexed form, e.g. with the complex immobilized to a solid phase, typically comprises a change in desorbing conditions for instance increasing the concentration of the dissociating agent or replacing the dissociating agent with another dissociating agent.

In principle any kind of measuring method (=assay) that can be used for measuring EPO can be used in step (iii) or in any other fraction containing isoEPOs. Preferably the measuring method is selected amongst various kinds of biospecific affinity assays. The formats of these assays are well known in the field. As applied to the measurement of EPO, they encompass that one or more affinity counterparts, for instance one or more antibody preparations specific for EPO are used for the formation of an affinity complex. The level of complex formation is then measured and related to the level of EPO in one or more of the fractions obtained in step (ii). The formats may or may not use an affinity reactant that is analytically detectable, e.g. labelled. Examples of suitable analytically reactants are labelled EPO or labelled antibody specific for EPO. The formats may or may not utilize an affinity reactant that is immobilized or immobilizable to a solid phase. The immobilized affinity reactant is descriptively called capturer or catcher. One way of grouping formats utilizing labelled reactants and/or immobilized or immobilizable reactants is in competitive and non-competitive assays. The sandwich format is a typical non-competitive format. It utilizes at least two affinity counterparts that in the invention are specific for EPO so that they simultaneously can bind this analyte. One of the counterparts typically exhibits a label while the other exhibits another label or is immobilized or immobilizable to a solid phase. The competitive formats typically utilize an EPO analogue, e.g. EPO in labelled form or in immobilized or immobilizable form. The analogue is typically competing with. EPO for binding to a common affinity counterpart (anti-EPO antibody) that is present in limiting amount. The affinity counterpart may be in labelled form in the case the EPO analogue is in immobilized or immobilizable form and in immobilized or immobilizable form if the EPO analogue is in labelled form. So called displacement assays are often considered as competitive formats. The formats may also be divided into heterogeneous and homogeneous formats where heterogeneous formats require a separation of labelled reactant incorporated in a complex from the same labelled reactant not incorporated in the complex before the labelled reactant is measured. Biospecific assay formats also include immunoblotting, agglutination formats (particles as labels), nephelometric/turbidometric formats etc.

Preferred biospecific affinity assays for EPO are typically heterogeneous and utilize typically an anti-EPO antibody that is immobilized or immobilizable to a solid phase. The assays comprise the step of forming an immobilized affinity complex that comprises EPO and anti-EPO antibody with immobilization to the solid phase via the anti-EPO antibody. Measurement of the amount of complex formed is typically by the use of labelled EPO or a labelled anti-EPO antibody.

A very interesting way of measuring is by mass spectrometry (MS) since this would facilitate measuring specifically desired isoEPOs even if they are present together with other isoEPOs, i.e. measurement of subpopulations containing very few isoEPOs (e.g. one, two, three etc isoEPOs).

In preferred variants the measurement is carried out on a fraction in which the analyte isoEPOs are in uncomplexed form, i.e. after being dissociated from the complex formed in step (ii.A). One can also envisage measurement on a fraction in which the analyte isoEPOs are complexed to the isoEPO reactant after irrelevant isoEPOs at least partly have been removed from the complex formed in step (ii.A). This latter variant typically requires a relatively strong affinity binding between the analyte isoEPOs and the isoEPO reactant and will be simplest to perform if the affinity complex is immobilized to a solid phase via the isoEPO reactant.

The EPO specific affinity assay used in step (iii) does not require a discrimination between analyte isoEPOs and other isoEPOs except for the case when the enrichment degree with respect to analyte isoEPOs in steps (iiA) to (ii.B) is poor (relative to other isoEPOs).

The solid phases used in biospecific assays are typically selected amongst the same solid phases as can be used as solid phases for the isoEPO reactant in step (ii). See above.

In flow systems of the type described above in which the isoEPO reactant is immobilized in a separation zone SZ and a capturer is immobilized in the detection zone DZ. The capturer is typically an anti-EPO antibody directed towards the polypeptide part of EPO. The affinity constant of a suitable capturer [e.g. (complex)/(capturer)(EPO)] is typically at least equal to or 10 or 10² or 10³ times larger than the corresponding affinity constant between the isoEPO reactant used in the separation zone and EPO/analyte isoEPO.

### The Flow Matrix Aspect of the Invention (2^{nd} Main Aspect)

This aspect is a flow matrix of the type described above containing an application zone and a downstream separation zone in which there is an immobilized EPO-specific affinity reactant. The characteristic feature is that the EPO specific affinity reactant is an isoEPO reactant as defined for the first aspect, preferably with carbohydrate specificity. The immobilization is preferably via a carrier molecule with further preference for the linkage between the carrier and the flow matrix comprising affinity binding and the linkage between the carrier and isoEPO reactant being covalent. The preferences for the carrier are the same as given for the method aspect. Other characteristics of the flow matrix aspect are the same as given for the flow matrix used in the method aspect of the invention.

### Best Mode

The examples given in the experimental part represent the best mode. A potentially very useful variant is represented in example 3. See also the preferred embodiments given above.

### EXPERIMENTAL PART

### Example 1

### Test for Measuring the Distribution of EPO Isoforms Using Chromatographic Separation of EPO by WGA Column and Gradient Elution with Competing Sugar Derivative

Sample material. Urine specimens were collected from normal individuals, and from patients receiving rhEPO or Aranesp. The EPO in each sample was affinity purified in accordance with common practise.

Separation of EPO using WGA. About 1 ml with 120-400 pg of affinity purified EPO from a urine specimen in a buffer containing 0.2 mg BSA/ml, 20 mM bis-tris pH 6.4, 0.1% tween 20, 0.02% NaN₃, 8 μM pepstatin (Sigma) and 1/500 protease inhibitor (Sigma P8340) was applied to a 0.9 ml column of WGA-Sepharose connected to an ÄKTAexplorer 10 automatic chromatography system (GE Helthcare, Uppsala, Sweden). The WGA-Sepharose was prepared by reacting WGA (Medicago, Uppsala, Sweden) with NHS-Sepharose (GE Helthcare, Uppsala, Sweden) by adding 4.9 mg WGA to one ml of sedimented gel in accordance with the instructions from the supplier. The chromatographic separation was performed with a start-buffer containing 20 mM TRIS pH 7.5, 0.15 M NaCl, 0.1% tween 20, 0.05% NaN₃, 1/500 protease inhibitor (Sigma P8340) and the gradient of competing sugar derivative was formed by mixing the start-buffer with the same buffer containing N-acetyl glucose amine (GlcNAc). The flow rate was 1 ml/min and sample application and washing with start-buffer was performed during 7 minutes. A linear gradient from 0 to 15 mM GlcNAc was formed during 10 minutes, 15 mM and 50 mM GlcNAc was applied during 2 and 3 minutes, respectively. Aliquots of 0.35 ml were collected in microtiter wells.

Measurement of EPO in the fractions by immunochromatographic test. The fractions were tested by an immunochromatographic EPO test where 50 μl of sample was dispensed in microtiter wells and a 5 mm wide and 22 mm long strip (MAIIA AB, compare U.S. Pat. No. 6,737,278 and US 20040023412 (Caisson & Lönnberg), with a thin line of anti-EPO 3F6 about 13 mm from one end of the membrane with the other end mounted on a 30 mm absorbent sink GB004, was placed in each well. After 5 min. the complete sample volume had been sucked up and the strip was moved into another well containing 25 μl of carbon black antiEPO 7D3 (MAIIA AB) in which it was left for 7 min. and finally placed into a well containing 20 μl washing solution (MAIIA AB) for 7 min.

The strips were mounted on a paper sheet, the absorbent sink was removed and the sheet was placed in a scanner after the strips had dried. The intensity of carbon black in the capturing anti-EPO zone was measured and delta blackness per pixel was calculated in accordance with earlier description [Anal. Biochem. 293, 224-231 (2002)].

The concentration of EPO in the sample was calculated against the delta blackness per pixel obtained when analyzing a dilution sequence of samples with known EPO.

### RESULTS

Calculation of the relative amount of EPO in each well. The total amount of EPO obtained in all the fractions was summed up and the percentage of total EPO in each fraction calculated. By presenting the results as % EPO per well, instead of pg per well which is in accordance with commonly used presentations using absorbance units it was possible to better compare the distribution when different amounts of EPO was applied on the column. In FIG. 1 is shown the separation profile obtained for four sample groups when summing up the figures (% EPO) after WGA separation of 9 normal urines, 3 rhEPO preparations and 2 Aranesp preparations as well as from 3 urines which seem to contain mainly an aberrant EPO form. It was possible to separate the different forms by utilizing a gradient elution with low concentration of a competing sugar derivative (N-acetyl glucose amine, GlcNAc). The EPO analogue Aranesp, with five carbohydrate structures, showed the highest affinity for WGA and required the largest volume before it was released and eluted. Recombinant EPO (rhEPO), like Eprex (Jansen-Cilag) and Neorecormone (Roche) had lower affinity compared to Aranesp but higher affinity when compared to normal endogenous kidney EPO. Some urine specimens from patients receiving rhEPO and Aranesp had an aberrant EPO form that had low affinity for WGA. Most probably this is an endogenous form of EPO, aberrant from the normal EPO produced in the kidney, present in the urine from patients suffering of anemia. One possibility is that the liver cells start to produce EPO due to insufficient production of EPO by the kidney in response to hypoxia or due to too low dose of rhEPO.

Many urine specimens contain EPO from two or several of the different EPO populations. Especially patients receiving rhEPO or Aranesp can show endogenous kidney forms, the aberrant forms and the recombinant forms in the same urine specimen. In FIG. 1 is also shown the isoform profile for a urine specimen (U33) from a patient receiving Aranesp, where both Aranesp and the aberrant EPO form can be seen.

However, from such isoform profile there is a need to calculate adequate figures that can give unambiguous information about the content of all the different isoform populations or of selected ones. If only two populations are available it is possible to use a figure for the mean elution volume. If all the populations are going to be quantified, pattern recognition has to be used to evaluate the obtained isoform profile. The presence of rhEPO or Aranesp, both illegally used as doping substances in endurance sports, and the presence of aberrant endogenous EPO can be identified by utilizing figures from the isoform profile.

Two populations—calculation of elution volume for 50% of EPO. The total amount of EPO obtained in the fractions was summed up and the elution volume for 50% of EPO for some specimens was calculated. It was found that four groups with distinct different 50% elution volumes could be distinguished. 50% of EPO had eluted at 13.5±0.22, 14.7±0.28, 16.7±0.62 and 11.3±0.12 for 9 normal urines, 3 rhEPO preparations, 2 Aranesp preparations and from 3 urines which seem to contain mainly an aberrant EPO form, respectively. Using this type of evaluation of the results, where only one figure is obtained to classify the type of EPO isoform population, is easier for interpretation. However, if there are more than two dominant isoform populations available or if two isoform populations are separated on each side of a third one it is not sufficient to characterize the composition with only one figure.

Calculation of the elution volume for 50% of a sample containing both Aranesp and the aberrant form (U33) will give a false value (14.9 ml) corresponding to rhEPO due to the mean value obtained from a large elution volume for Aranesp and a small elution volume for the aberrant form. Such samples require the calculation of two figures characteristic for each population.

Determination of all isoform populations by pattern recognition. By comparing the obtained isoform profile (% EPO per fraction) from a sample to the pattern obtained when simulating a mixed composition by the figures obtained from pure isoform populations, as shown in FIG. 1, it was possible to calculate the proportions of isoform populations in the patient urine samples. Urine from patients receiving rhEPO showed in average 35% EPO eluted at the same position as EPO produced in the kidney, and 65% eluted at the same position as rhEPO. One urine specimen from a patient receiving Aranesp showed 25% EPO eluted at the same position as EPO produced in the kidney, and 75% eluted at the same position as Aranesp. One urine from a patient receving rhEPO showed 100% of EPO eluted at the position for the aberrant EPO, while four urines from patients receiving Aranesp showed 84% EPO eluted in that position and the remaining 16% in the position for Aranesp. The three remaining urines from patients receving Neorecormon had EPO that eluted in the positions for EPO produced in the kidney, rhEPO and aberrant EPO.

Selection of figures for identifying one isoform population. The presence of rhEPO or Aranesp, which both are illegally used as doping substances in endurance sports, was identified by calculating the sum of % EPO for the elution volumes between 18.9 to 20.3 ml (D1) and the sum of % EPO between 12.95 to 13.65 ml (D2) from the isoform profile and calculating the ratio D1/D2. EPO from normal urine showed values of 0.41±0.19 (mean±2 standard deviations). Above 0.60 the samples were regarded as positive for doping and patients receving rhEPO or Aranesp, without indications of aberrant EPO, showed an average ratio value of 1.13 (range 0.47-2.66) while eight patients with indications of aberrant EPO showed an average ratio value of 0.98 (range 0.29-3.31).

It was also possible to get an “aberrant EPO” factor by summing up the amount EPO in fractions from 9.1 to 9.8 ml (L1) and between 12.6 to 13.3 ml (L2) and calculating a ratio of L1/L2. EPO from normal urines showed values of 0.13±0.06 (mean±2 standard deviations). Above 0.19 the samples were regarded as containing aberrant EPO and eight patients showed ratio values in the range 0.21-1.56.

### Example 2

### Test for Measurement of EPO Isoform Populations by Step-Wise Elution from the WGA Column

Sample material. Urine specimens were collected from normal individuals and from patients receiving rhEPO or Aranesp. The EPO in each sample was affinity purified in accordance with common practise.

Separation of EPO using WGA. About 0.8 ml with 100-1000 pg of affinity purified EPO from urine specimens in a buffer containing 17 mM bis-tris pH 6.4, 0.1% tween 20 0.02% NaN₃ were applied to a Pasteur-pipette containing 0.39 ml of WGA-Sepharose (GE Helthcare, Uppsala, Sweden).
The chromatographic separation was performed with an elution buffer containing 20 mM TRIS pH 7.45, 0.15 M NaCl, 0.1% tween 20, 0.05% NaN3 and the same buffer with 2.5, 5, 20 and 500 mM N-acetyl glucose amine (G1cNAc) added. The sample was applied and 2.8, 2.4, 2.4, 2 and 2.4 ml of 0, 2.5, 5, 20 and 500 mM GlcNAc buffer, respectively, were consecutively applied to the column. Aliquots of eluate (about 0.4 ml) were collected in microtiter wells.

Measurement of EPO in the fractions by immunochromatographic test. The measurement of the fractions by the immunochromatographic EPO test was performed in accordance with the description in Example 1.

### RESULTS

Calculation of the relative amount of EPO in each well. In FIG. 2 is shown the separation profile, in % EPO per fraction, for the three groups obtained when summing up the figures after WGA separation of 5 normal urines, 4 rhEPO preparations and one Aranesp preparation. The three isoform populations show distinguishable isoform profiles. The results obtained by urine from a patient receiving Aranesp (U33), show an aberrant profile with one early eluting isoform population, probably containing aberrant endogenous EPO, as well as Aranesp which elutes late.

Calculation of figures from the elution profile. It is possible to obtain figures from the elution profile which can be used for statistical calculations that can be used to classify the populations of EPO isoforms in the sample. Samples containing only one isoform population can easily be distinguished by calculating the % EPO from selected elution steps or the sum of several steps. However, for the urine from the patient receiving Aranesp (U33), who also has the aberrant EPO isoform, the presence of Aranesp can be recognised only by calculating a ratio between EPO in the fraction-steps containing 20 and 5 mM GlcNAc as the aberrant EPO affects the percentage distribution. In this sample aberrant EPO was easily identified by the % EPO which eluted early in the 0 mM GlcNAc fractions, but the calculation of the ratio of EPO in the 0 and 2.5 mM GlcNAc fractions will be required if higher proportions of Aranesp or rhEPO is present.

### Example 3

### Test for Measuring the Presence of EPO-Resembling Doping Substances and Presence of Aberrant EPO by a EPO WGA MAIIA Teststrip

Sample material. Urine specimens were collected from normal individuals and from patients receiving rhEPO or Aranesp. The EPO in each sample was affinity purified in accordance with common practise.

WGA-BSA conjugate. WGA (Medicago, Uppsala, Sweden) and BSA (Intergen Company, NY, USA) were reacted with SPDP (N-Succinimidyl 3-(2-pyridyldithio)propionate, Pierce, Rockford, Ill., USA) in the molar proportions 1:1.5 and 1:5, respectively, in accordance with the instructions from the supplier.

The obtained BSA-2-pyridyldisulphide derivative was then incubated with dithiotreitol to form BSA-SH which, after a desalting step, was reacted with a WGA-2-pyridyldisulphide derivative in the molar proportions 4:1, during 24 h incubation, forming a conjugate between WGA and BSA.

WGA-MAIIA strip. Nitrocellulose membrane (Whatman Int. Ltd, Maidstone, UK) with 3 μm nominal pore size and backed with an optically clear polyesterfilm was cut into 3.5×30 cm sheets. Lines with WGA-BSA, 1.5 mg WGA/ml and anti-EPO 3F6 (MAIIA AB, Uppsala, Sweden), 1 mg/ml, were deposited with 1 μl/cm along the sheet using a Frontline dispensor (Biodot Inc. Irvine, Calif., USA). The sheet was mounted with a 3×30 cm absorbent sink GB004 (Schleicher and Schuell GmbH, Dassel, Germany) using tape and finally cut into 5 mm wide strips by using a cutting module (Biodot Inc.), resulting in 6 cm long strips. The positions of the WGA-BSA lines (5 lines over 5 mm) and anti-EPO (1 line, 1 mm wide) is shown in FIG. 3a. Compare U.S. Pat. No. 6,737,278 and US 20040023412 (Carlsson & Lönnberg)

EPO WGA MAIIA test procedure. The affinity purified samples were diluted in 20 mM TRIS pH 7.5, 150 mM NaCl, 0.1% Tween 20, 0.05% NaN₃, protease inhibitor P8340, 1/500, 10 μM Pepstatin and 0.5 mM EDTA. 30 μl of diluted sample was dispensed into a microtiter well where a WGA-MAIIA strip was placed and left for 5 min. After all the sample had been sucked up, the strip was placed in a well containing 25 μl of elution buffer with 8 or 40 mM GlcNAc, 20 mM TRIS pH 7.5, 150 mM NaCl, 0.1% Tween 20, 0.05% NaN₃, and left for 5 min. The WGA zone of the strip was cut of and the remaining strip with the anti-EPO zone was placed in a well with 25 μl of carbon black anti-EPO 7D3 (MAIIA AB) and left for 5 min. Finally, the strip was placed in a well with 25 μl washing solution (MAIIA AB) and left for 5 min.

The strip was mounted and measured by the scanner as described in Example 1.

The concentration of EPO in the sample was calculated by comparing the obtained delta blackness per pixel with the values obtained when measuring a dilution sequence of samples with known EPO, using strips containing only anti-EPO capturing line. Calculating the ratio of the EPO concentration obtained when using 8 mM GlcNAc/the EPO concentration obtained when using 40 mM GlcNAc gave the % EPO passing the WGA zone, as when using 40 mM GlcNAc all EPO was released from the WGA zone on the strip.

Results. When using 8 mM GlcNAc for elution of bound EPO forms, the Aranesp forms, represented by Aranesp added to low urine and urine from a patients receiving Aranesp, had the strongest affinity to WGA and only 28% was released when 8 mM GlcNAc was used for elution. For rhEPO forms, represented by urine from patients receiving rhEPO and a urine with added rhEPO, 41% was released, while 72% of total EPO was released when testing normal urines. The group with urine from patients that had the aberrant form released 97% of all EPO when 8 mM GlcNAc was used for elution.

The optimal concentration of GlcNAc depends on the available concentration of WGA on the membrane, the length of the flow path and the required sensitivity both for detecting doping as well as for detecting the presence of the aberrant EPO form. It was also convenient to use a set of strips where different concentrations of sugar were utilized for elution. In that case better sensitivity was obtained for revealing the presence of the aberrant form by using lower concentration than 8 mM GlcNAc whereas higher sugar concentration was optimal for doping detection.

### FIGURES

FIG. 1. Separation profile of EPO isoforms using WGA column.

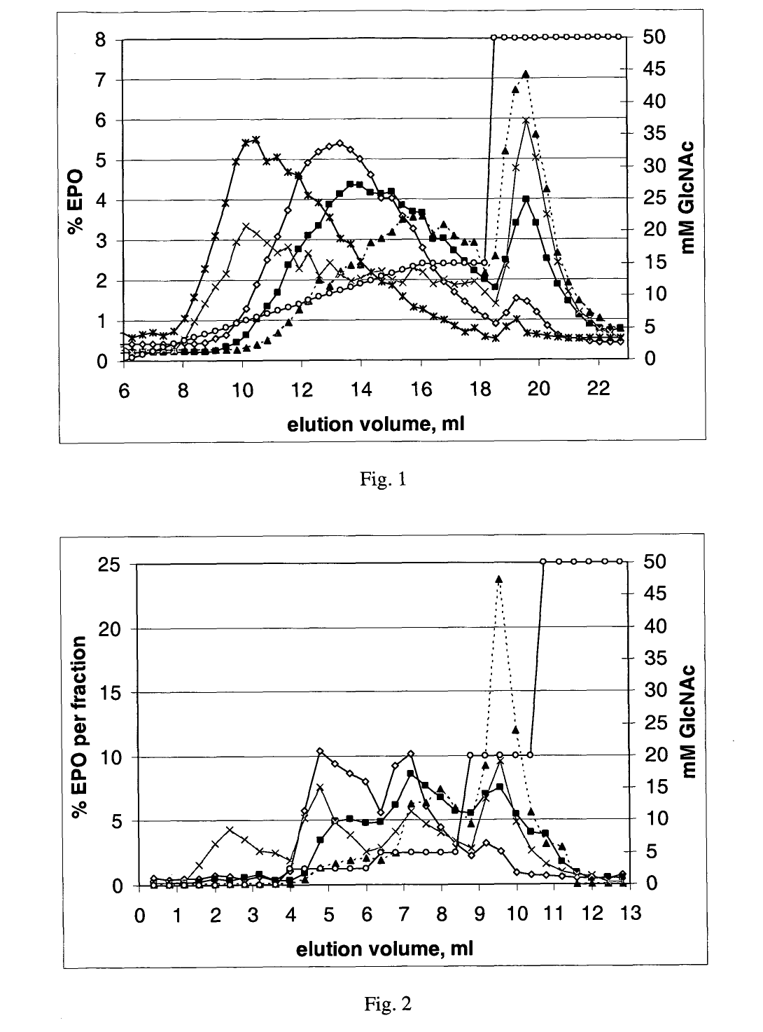

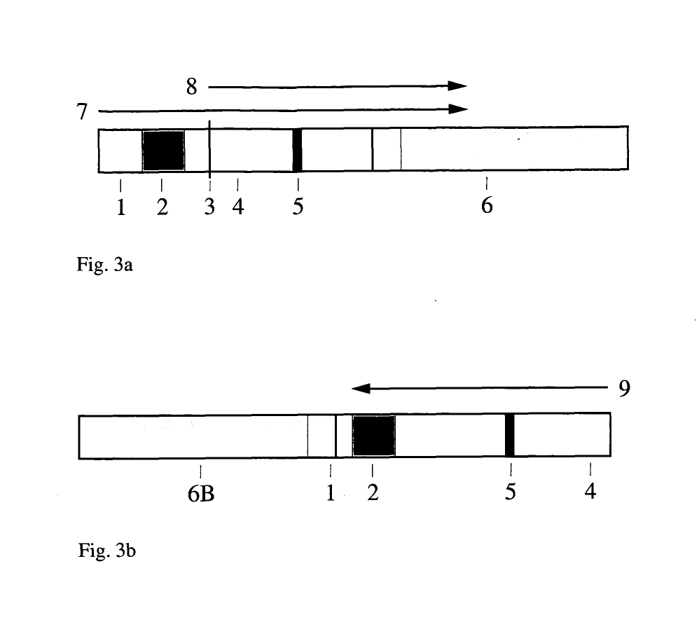

The percentage of EPO in each fraction was calculated and plotted against elution volume. The results for 9 urines with normal kidney EPO, 3 rhEPO preparations and 2 Aranesp preparations were compiled. Results from 3 urines were also compiled as they all showed an aberrant form which eluted very early. It was possible to separate these different forms by a gradient elution with low concentrations of competing sugar (G1cNAc).

Patient urine U33 showed two distinct isoform populations, Aranesp and the aberrant EPO form with low affinity for WGA.

Legends: ⋄ Endogenous kidney EPO, ▪ Recombinant EPO, ▴ Aranesp, [symbol] Aberrant endogenous EPO, x urine U33, ∘ mM GlcNAc.

FIG. 2. Stepwise elution of EPO isoforms from WGA column using increasing concentrations of competing sugar derivative.

After calculation of % of EPO in each fraction of 5 normal urines, 4 rhEPO preparations and one Aranesp preparation after separation, distinguishable isoform profiles were obtained. The figures for the normal urines as well as for the rhEPO preparations were compiled showing that endogenous kidney EPO elutes earlier than rhEPO from the column. The results for a patient urine (U33) containing both aberrant EPO, which was eluting early, and Aranesp, which was eluting late, is also shown.

Legends: ⋄ Endogenous kidney EPO, ▪ Recombinant EPO, ▴ Aranesp, x urine U33, ∘ mM GlcNAc.

FIG. 3a. WGA-MAIIA strip

From the left in the figure is shown the sample application zone (1), the 5 mm wide separation-zone with 5 lines with WGA-BSA (2) applied 5 mm up on the membrane, the cutting line (3) at position 13 mm, the application zone for the detectable reactant (5), the detection zone with the one mm wide antibody line at position 23 mm (5) and the adsorbent sink (6). The flow paths for sample and desorption reagent (7) and for the detectable reactant (8) are indicated by arrows. Compare U.S. Pat. No. 6,737,278 and US 20040023412 (Carlsson & Lönnberg)

FIG. 3b. Opposite flow path

The detectable reagent can also be applied on alternative positions and a flow direction opposite to the sample and desorption flow path, shown in FIG. 3a, can be used. After performing the desorption flow (7), as illustrated in FIG. 3a, the first absorbent sink is removed and another absorbent sink (6B) is placed on the sample application position (1). The detectable reactant is then applied (4) and a flow path (9) in opposite direction is obtained.

While the invention has been described and pointed out with reference to operative embodiments thereof, it will be understood by those skilled in the art that various changes, modifications, substitutions and omissions can be made without departing from the spirit of the invention. It is intended therefore that the invention embraces those equivalents within the scope of the claims which follow.

1. A method for the determination of the occurrence of an analyte subpopulation of isoerythropoietins (isoEPOs in a biological fluid comprising the steps of:
i) providing a liquid sample deriving from the biological fluid and containing isoEPOs that are characteristic (analyte isoEPOs) for said analyte subpopulation together with isoEPOs of other subpopulations (other isoEPOs),
ii) separating the isoEPOs of said liquid sample into two or more fractions so that at least one of these fractions contains analyte isoEPOs which are enriched relative to other isoEPOs by
A) contacting the liquid sample with an isoEPO reactant, which is capable of discriminating between analyte isoEPO and other isoEPOs, under conditions allowing affinity complex formation between isoEPOs of the liquid sample and the affinity reactant, and
B) fractionating and collecting the isoEPOs of the liquid sample into said two or more fractions, and
iii) relating the occurrence of analyte isoEPOs in said at least one fraction to the occurrence of said analyte subpopulation in the biological fluid.

2. The method of claim 1, wherein step (iii) comprises the steps of:
iii.a) measuring the amount of said analyte isoEPOs that are present in one or more of said at least one fraction, and
iii.b) relating the amount found in step (iii.a) to the occurrence of said analyte subpopulation in the biological fluid.

3. The method of claim 2, wherein the amount measured in step (iii.a) is relative to the total amount of isoEPOs in the liquid sample and/or relative to the amount of isoEPOs of one or more fractions that are different from said at least one fraction that is enriched in analyte isoEPOs, said one or more fractions preferably being enriched in isoEPOs that are characteristic for at least one subpopulation other than the analyte subpopulation and being comprised within said two or more fractions.

4. The method of claim 1, wherein said analyte subpopulation is exogenous to the mammal, e.g. recombinant EPO, such as a recombinant variant having a different carbohydrate composition or structure compared to endogenous EPO and/or a mutated variant of endogenous EPO.

5. The method of claim 1, wherein said analyte subpopulation is endogenous, e.g. of renal or hepatic origin, and/or disease-related.

6. The method of claim 1, wherein said isoEPO reactant exhibits biospecific affinity for a carbohydrate structure, the exposure of which is different in analyte isoEPOs compared to in other isoEPOs, typically said reactant being a lectin including also an anti- carbohydrate antibody, for instance with said reactant being wheat germ agglutinin (WGA) and/or said carbohydrate structure exhibiting N-acetyl glucose amine structure.

7. The method of claim 1, wherein step (ii.A) comprises that said affinity complex is formed in insolubilized form, i.e. with said iso-EPO specific affinity reactant being in insoluble form prior to the formation of said affinity complex, e.g. immobilized to a solid phase.

8. The method of claim 7, wherein step (ii.B) comprises the steps of:
a′) adjusting the conditions provided by the liquid phase in contact with the immobilized affinity complex such that analyte isoEPOs are selectively dissociated/released from or selectively retained on the solid phase thereby enriching analyte isoEPOs to the liquid phase in uncomplexed form or to the solid phase in complexed form, respectively, and
b′) collecting the isoEPOs complexed on the solid phase or uncomplexed in the liquid phase as said at least one fraction enriched in analyte isoEPOs.

9. The method of claim 8, wherein step (ii.B. a) comprises the step of:
a″) adjusting the conditions provided by the liquid phase in contact with the solid phase to which the affinity complex is immobilized such that isoEPOs of none, one, two, three or more subpopulations other than said analyte subpopulation are dissociated/released from the complex/solid phase subsequently and/or prior to the dissociation and capturing of analyte isoEPOs, and
b″) collecting these other released isoEPOs as the remaining ones of said two or more fractions.

10. The method of claim 7, wherein steps (ii.A) and (ii.B) at least partially coincide and comprise the steps of:
a) adjusting the conditions in the liquid phase/sample to promote selective complexation of analyte isoEPOs or of non-analyte isoEPOs with the immobilized isoEPO reactant thereby enriching analyte isoEPOs to the solid phase in complexed form or to the liquid phase in uncomplexed form, respectively, and
b) collecting the isoEPOs complexed on the solid phase or uncomplexed in the liquid phase as said at least one fraction enriched in analyte isoEPOs.

11. The method of claim 1, wherein said isoEPO reactant is immobilized or immobilizable to a solid phase, e.g. comprising a porous matrix, selected amongst
a) inner walls of vessels in which complex formation is going to take place, and
b) porous beds.

12. The method of claim 11 , wherein
a) step (ii.A) comprises that the solid phase is a flow matrix and transporting the dissolved isoEPOs through the matrix by a liquid flow, and
b) steps (ii.B. a) and (ii.B. a′) comprise the step of adjusting the conditions provided by the liquid flow by including a desorption agent of constant or increasing concentration subsequent to the formation of the immobilized affinity complex.

13. The method of claim 12, wherein step (iii) comprises that EPO is measured by the use of a biospecific affinity assay for EPO, typically a heterogeneous variant e.g. utilizing anti-EPO antibody immobilized or immobilizable to a solid phase, by forming an immobilized affinity complex comprising EPO and an affinity counterpart to EPO in which immobilization to the solid phase is via the affinity counterpart, and measuring the amount of complex formed, e.g. by the use of labelled EPO or a labelled affinity counterpart to EPO.

14. The method of claim 13, wherein
A) the solid phase used in step (ii) for obtaining said at least one fraction in which the analyte subpopulation is enriched and the solid phase used in step (iii) for measuring the amount of EPO in said at least one fraction are in liquid flow communication with each other with the latter solid phase defining a detection zone downstream of the former solid phase that defines a separation zone, preferably with the two zones being part of a common porous matrix typically extending between the zones,
B) step (ii) comprises transporting said analyte isoEPOs that are desorbed in step (ii) into the detection zone where they are captured, and
C) step (iii) comprises that the amount of EPO that is captured in the detection zone is measured by the use of an analytically detectable reactant such as labelled EPO or labelled affinity counterpart to EPO.

15. The method according to claim 14, wherein said detectable reactant is applied to said porous matrix in
A) in an application zone between the sample application zone and the detection zone, i.e. transported in a flow path 2 that coincides with the downstream part of the flow path 1 between the sample application zone and the detection zone, or
B) in an application zone that together with the detection zone defines a flow path 2′ that is separate from the flow path 1 between the sample application zone and the detection zone, e.g. is transversal or has an opposite flow direction compared to the flow direction of flow path 1
between the sample application zone and the detection zone, or C) in a application zone that coincides with the detection zone with transport out of the zone of excess labelled reactant after reaction.

16. The method according to claim 13, wherein desorption of isoEPOs other than said analyte isoEPOs is done by a desorption liquid passing through the separation zone in a direction that is transversal to the flow direction in the separation zone, in particular for isoEPOs that are desorbed prior to analyte isoEPOs.

17. The method according to claim 13, wherein the separation zone is removed from the common matrix subsequent to desorption from the separation zone of isoEPOs other than said analyte isoEPOs but before applying the detectable reactant to the common matrix.

18. A flow matrix containing an application zone and downstream thereof a separation zone in which there is an immobilized isoEPO reactant.

19. The flow matrix of claim 18, wherein the affinity reactant exhibits carbohydrate specificity.

20. The flow matrix of claim 18, wherein the immobilization is via a carrier, preferably having polymer structure and preferably with the linkage between the carrier and the flow matrix comprising affinity binding and the linkage between the carrier and the isoEPO reactant being covalent.

21. The method of claim 2, wherein step (iii) comprises that EPO is measured by the use of a biospecific affinity assay for EPO, typically a heterogeneous variant e.g. utilizing anti-EPO antibody immobilized or immobilizable to a solid phase, by forming an immobilized affinity complex comprising EPO and an affinity counterpart to EPO in which immobilization to the solid phase is via the affinity counterpart, and measuring the amount of complex formed, e.g. by the use of labelled EPO or a labelled affinity counterpart to EPO.
